(19) United States
(12) Patent Application Publication
(10) Pub. No.: US 2013/0109977 A1
(43) Pub. Date: May 2, 2013

(54) UV IMAGING FOR INTRAOPERATIVE TUMOR DELINEATION

(71) Applicants: Shouleh Nikzad, Valencia, CA (US); Michael E. Hoenk, Valencia, CA (US); Todd J. Jones, Altadena, CA (US); Samuel R. Cheng, Walnut, CA (US)

(72) Inventors: Shouleh Nikzad, Valencia, CA (US); Michael E. Hoenk, Valencia, CA (US); Todd J. Jones, Altadena, CA (US); Samuel R. Cheng, Walnut, CA (US)

(73) Assignee: California Institute of Technology, Pasadena, CA (US)

(21) Appl. No.: 13/666,915

(22) Filed: Nov. 1, 2012

Related U.S. Application Data
(60) Provisional application No. 61/554,122, filed on Nov. 1, 2011.

(51) Int. Cl. A61B 5/00

(52) U.S. Cl. CPC A61B 5/0077
USPC 600/476

(57) ABSTRACT

A medical imaging system and method. A UV/visible camera uses a back illuminated silicon imaging detector to observe a surface of a brain of a human subject in vivo during brain surgery for excision of a cancerous tumor. The detector can be a CCD detector or a CMOS detector. Under UV illumination, the camera can record images that can be processed to detect the location and extent of a cancerous tumor because the presence of auto-fluorescent NADH variations can be detected between normal and cancerous cells. The image data is processed in a general purpose programmable computer. In some instances, an image is also taken using visible light, and the identified cancerous region is displayed as an overlay on the visible image.

### CROSS-REFERENCE TO RELATED APPLICATIONS

This application claims priority to and the benefit of co-pending U.S. provisional patent application Ser. No. 61/554,122 filed Nov. 1, 2011, which application is incorporated herein by reference in its entirety.

### STATEMENT REGARDING FEDERALLY FUNDED RESEARCH OR DEVELOPMENT

The invention described herein was made in the performance of work under a NASA contract, and is subject to the provisions of Public Law 96-517 (35 USC 202) in which the Contractor has elected to retain title.

### THE NAMES OF THE PARTIES TO A JOINT RESEARCH AGREEMENT

Caltech—JPL and Cedars Sinai Medical Center located in Los Angeles, Calif. have an agreement under which the camera of the invention is used under operating room conditions.

### FIELD OF THE INVENTION

The invention relates to cameras in general and particularly to a camera system and methods used to detect cancerous cells.

### BACKGROUND OF THE INVENTION

Computed tomography (CT), magnetic resonance imaging (MRI), positron emission tomography (PET) and ultrasound mostly provide pre and post operative information about the degree of invasiveness and location of a tumor. CT and MRI involve the use of intravenous contrast reagents to co-localize the brain tumor location. However 31% of anaplastic astrocytomas (intermediate-grade) and 4% of glioblastoma multiforme (high grade) do not appear enhanced on CT and or MRI even after contrast reagent is injected into the patient. See NA Rehua, et. al, “Can Autofluorescence Demarcate Basal Cell Caricinoma from Normal Skin? A Comparison with Protopiohyrin IX Fluorescence, Acta Derm Venereol 81:246-49, 2001. On the other hand, PET scans cannot be used intraoperatively. Ultrasound is limited by signal artifacts caused by blood and surgical trauma at the resection margin once the resection starts.

Fluorescence dyes, time resolved laser induced fluorescence spectroscopy and laser-induced fluorescence of the photosensitizer, hematoporphyrin derivative also has been used to delineate various tumors of the lung, the bladder, the colon and the brain from normal tissue. See NA Rehua, et. al, “Can Autofluorescence Demarcate Basal Cell Caricinoma from Normal Skin? A Comparison with Protopiohyrin IX Fluorescence, Acta Derm Venereol 81:246-49, 2001; B. W. Chwriot, et. al., “Laser-induced fluorescence: experimental intraoperative delination of tumor resection margins,” Journal of Neurosurgery 76:679-86, 1992; B. W. Chwriot, et. al., “Detection of Melanomas by Digital Imaging of Spectrally Resolved Ultraviolet Light-induced Autofluorescence of Human Skin,” European Journal of Cancer 34:1730-34, 1998; Wei-Chiang Lin, Steven A. Toms, Massoud Motamedi, Duco Jansen, Anita-Mandevan-Jansen, “Brain tumor Demarcation using optical spectroscopy; an in vitro study,” Journal of Biochemical Optics 5(2), 214-220 (April 2000); Jui Chang Tsai et al, “Fluorospectral study of the Rat Brain and Glioma in vivo,” Laser in Surgery and Medicine 13:321-331 (1993); Reid C. Thompson, M. D., Keith L. Black, M. D., Babek Kateb, Laura Marcu, Ph.D., “Time-Resolved Laser-Induced Fluorescence Spectroscopy for detection of Experimental Brain Tumors,” Congress of Neurological Surgeons, San Deigo, Calif., September 2001 oral/poster presentation; and Reid Thompson, Thanassis Papaioannov, Babak Kateb, Keith Black, “Application of LASER spectroscopy and Thermal Imaging for detection of brain tumors,” American Association of Neurological Surgeons, April 2001, Toronto, Canada, abstract/poster. Another paper in the same field is Reid C. Thompson, Keith L. Black, Babek Kateb, Laura Marcu, “Detection of experimental brain tumors using time-resolved laser-induced fluorescence spectroscopy,” Optical Biopsy IV, Robert R. Alfano, Editor, Proceedings of SPIE 8 Vol. 4613 (2002) pp 8-12. The apparatus in the 2002 SPIE paper uses a monochromator and a multi-channel plate photomultiplier tube as a detector.

Unfortunately, in the case of photosensitizers the photosignaling is dependent on the injection of specific light sensitive compounds. In the case of time resolved laser induced fluorescence spectroscopy it can take a long time to acquire data from the tissue. Therefore, none of these techniques by themselves are found to be helpful for tumor delineation in a surgical environment. Use of laser-induced fluorescence attenuation spectroscopy (LIFAS) for detection of brain tumors is still under development and clinical analysis and is not ready to be used intraoperatively.

Yu, Q. and Heikal, A. A., “Two-photon autofluorescence dynamics imaging reveals sensitivity of intracellular NADH concentration and conformation to cell physiology at the single-cell level”, Journal of Photochemistry and Photobiology B: Biology 95 (2009) 46-57, describe the use of confocal microscopy using femtosecond laser pulses and photomultiplier tubes to perform time-resolved single photon counting detection, in which the discrimination between normal cells and cancer cells is based on differences of fluorescence lifetime. The observations were made on breast cancer (Hs578T) and normal (Hs578Bst) cells for quantitative analysis of the concentration and conformation (i.e., free-to-enzyme-bound ratios) of the NADH coenzyme. The samples measured were cells cultured in the lab, and not in vivo specimens.

There is a need for improved systems and methods for imaging cancerous regions in vivo.

### SUMMARY OF THE INVENTION

According to one aspect, the invention features a medical imaging system. The medical imaging system comprises a camera having a back illuminated silicon imaging detector that is sensitive in the visible and in the UV portions of the electromagnetic spectrum, the camera having a filter with a pass band in the UV, the filter being controllable to allow the camera to receive UV without visible illumination when the filter is engaged, and being controllable to allow the camera to receive visible illumination when the filter is removed from an optical path, the camera having at least one control port and having at least one output port; as required if not already present in a location of use, a UV illumination source configured to illuminate the field of operation in a surgical procedure on a brain of a patient, the UV illumination source, if provided, controllable by a controller; a controller configured to control an operation of the camera having the detector and the filter with the pass band in the UV; and a general purpose programmable computer configured to receive output data from the at least one output port of the camera having the detector, the general purpose programmable computer having access to instructions recorded on a machine readable medium, such that when the instructions are operating, the computer is programmed to operate the camera, record the data taken by the camera, process the data taken by the camera, and display a result of such computation to a user of the medical imaging system.

In one embodiment, the back illuminated silicon imaging detector is a detector selected from the group consisting of a δ-doped detector and a multilayer doped detector.

In yet another embodiment, the back illuminated silicon imaging detector is a detector having a device structure selected from the group consisting of a CCD detector, a CMOS detector, a photodiode detector array, a hybrid photodiode detector array, and an avalanche photodiode detector array.

In a further embodiment, the back illuminated silicon imaging detector comprises an anti-reflection coating.

In yet a further embodiment, the back illuminated silicon imaging detector has at least 1024×1024 pixels.

In an additional embodiment, the back illuminated silicon imaging detector is sensitive to illumination in the range of 420 nm-480 nm.

In one more embodiment, the UV filter blocks light in the wavelength ranges of 250 nm-442 nm and 498 nm-640 nm.

In still a further embodiment, the controller is implemented in the general purpose programmable computer.

According to another aspect, the invention relates to a method of detecting cancerous brain tissue in a human subject in vivo. The method comprises the steps of observing under UV illumination at least one image of a region of a surface of a brain of a human subject in vivo with a medical imaging system having a back illuminated silicon imaging detector as described hereinabove; recording the at least one image observed under UV illumination; processing in a general purpose programmable computer operating using instructions recorded on a machine readable medium the at least one image observed under UV illumination to determine a result, the result being a region within the image that is representative of a cancerous tumor; and performing at least one of recording the result, transmitting the result to a data handling system, or to displaying the result to a user of the medical imaging system.

In one embodiment, the method further comprises the steps of observing under visible illumination the region of a surface of a brain in a human subject in vivo to obtain a visible image; and using the visible image in displaying the result to the user.

In another embodiment, the UV illumination is in the range of 310 nm-415 nm.

In yet another embodiment, the UV illumination has a wavelength centered around 385 nm.

In still another embodiment, the UV illumination has a wavelength centered around 405 nm.

In a further embodiment, the back illuminated silicon imaging detector is a detector selected from the group consisting of a δ-doped detector and a multilayer doped detector.

In an additional embodiment, the back illuminated silicon imaging detector is detector having a device structure selected from the group consisting of a CCD detector, a CMOS detector, a photodiode detector array, a hybrid photodiode detector array, and an avalanche photodiode detector array.

In still a further embodiment, the back illuminated silicon imaging detector has an antireflection coating.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1 is a schematic flow diagram of an imaging process according to principles of the invention.

FIG. 2 is an image of a δ-doped CCD detector with a structurally supported membrane, which is advantageous for robustness and applications using fast optics.

FIG. 3 is an image of a portable, high-frame rate UV/visible camera.

FIG. 4 is an image of a piece of paper brushed with SPF 60 sunscreen recorded using room light as the illumination source.

FIG. 5 is an image of a piece of paper brushed with SPF 60 sunscreen recorded using a 385 nm LED as the illumination source.

FIG. 6 is an image of a piece of paper brushed with SPF 60 sunscreen recorded using both room light and a 385 nm LED as the combined illumination source.

FIG. 7 is a diagram that illustrates the distance and angular relationships between the camera and the surface to be viewed and recorded.

FIG. 8A illustrates Euremalisa butterflies imaged with the camera of FIG. 3 in ultraviolet.

FIG. 8B illustrates Euremalisa butterflies imaged with the camera of FIG. 3 in the visible.

FIG. 9A illustrates a rock observed under visible light illumination.

FIG. 9B illustrates the rock of FIG. 9A observed under UV illumination.

FIG. 10A illustrates an exposed region of a brain observed under visible light illumination.

FIG. 10B illustrates the exposed region of a brain of FIG. 10A observed under UV illumination.

FIG. 11 is a schematic diagram of an experimental observation of a human finger under UV illumination.

FIG. 12 is an image of a UV LED.

FIG. 13 is an image of a human finger observed under UV illumination conditions.

### DETAILED DESCRIPTION

Ultraviolet imaging (UVI) of brain tissue is expected to be a useful tool for intraoperative delineation of tumor resection margins. One of the significant variables affecting the survival and quality of life of patients with gliomas is the completeness of tumor resection. Although recent advances in neuroimaging have opened a new window of opportunity for neurosurgeons to obtain more extensive information regarding the location, the invasiveness and metabolic properties of brain tumors, current techniques still cannot provide real-time intraoperative feedback about the completeness of the resection.

Intraoperative use of a UV camera according to principles of the invention are expected to provide a tool for neurosurgeons to achieve 100% tumor resection. Previous experimental studies have provided significant capability of similar techniques involving UV observations with other instruments in enhancing optical imaging of human skin cancer, as described in the Rehua paper cited hereinabove.

One of the differences in treating brain cancer from other cancers is the well-known blood-brain barrier. The blood-brain barrier is a separation of circulating blood from the brain extracellular fluid in the central nervous system. It occurs along all capillaries and consists of tight junctions around the capillaries that do not exist in normal circulation. It is believed that the purpose of the blood-brain barrier is to protect the brain from hazardous materials that might pose dangers to the brain. The blood-brain barrier can therefore represent a problem in the treatment of brain cancers with drugs that are prevented from crossing the barrier. This is different from other situations in the body, where such exclusion of drug molecules is not a problem. Endothelial cells restrict the diffusion of microscopic objects (e.g., bacteria) and large or hydrophilic molecules into the cerebrospinal fluid, while allowing the diffusion of small hydrophobic molecules, such as O₂, CO₂, and hormones. Cells of the barrier actively transport metabolic products such as glucose across the barrier with specific proteins.

Nicotinamide Adenine Dinucleotide Hydrogenase (NADH) is a naturally occurring co-enzyme with auto-fluorescent peak excitation and emission at 340 and 480 nm respectively. This co-enzyme is up regulated in cancerous tissue (i.e., skin cancer) which could be detected by use of an ultraviolet/optical camera. The up-regulation is believed to be caused by changes in metabolic activities in cancerous vs. normal cells. It is believed that in vivo imaging approaches are useful to distinguish tumorous brain cells from normal brain tissue. In embodiments of the systems and method of the invention, one expects to generate a metabolic map of the tumor that is expected to differentiate it from the normal surrounding brain tissue. The imaging technology (using a special type of UV/Optical camera) plays a significant role in assisting neurosurgeons to delineate the tumor margins.

The approach taken to intraoperatively evaluate brain tumors for reflection/fluorescence changes between tumor and normal brain involves the use of a high-resolution UV camera (1 k×1 k) that is to be placed near to the surgical field so as to record images during tumor exposure and resection. It is expected that detectors having more pixels, such as an array of 1.5 k×2 k pixels, can also be used.

The detector used in the UV/Visible camera is in general a back illuminated silicon detector. In some embodiments, the detector is a back illuminated silicon detector passivated by molecular beam epitaxy (MBE). In other embodiments, the detector is any silicon detector passivated by delta doping or multilayer doping as detailed in the Table given below.

TABLE I
Detector Embodiments

| | Back Surface Passivation | |
|---|---|---|
| Device Structure | Delta doped | Multilayer Doped |
| CCD | Delta doped CCD | Multilayer doped CCD |
| CMOS | Delta doped CMOS | Multilayer doped CMOS |
| | Imaging Array | Imaging Array |
| Photodiode | Delta doped | Multilayer doped |
| Array | Photodiode Array | Photodiode Array |
| Hybrid photodiode | Delta doped Hybrid | Multilayer doped Hybrid |
| Array | Photodiode Array | Photodiode Array |
| Avalanche photodiode | Delta doped avalanche | Multilayer doped |
| array | photodiode array | avalanche |
| | | photodiode array |

In some embodiments, any of the above combinations can also be antireflection coated (AR coated) for further response enhancement in the UV.

It is expected that normal brain cells and tumor cells will be distinguished based on the differences in the NADH concentration present in them. It is believed that the use of this noninvasive technology intraoperatively will provide a tool to better assess the margins of the tumor and help neurosurgeons to contribute to the survival and quality of life of patients with malignant brain tumors.

In one embodiment, a delta doped CCD camera has been demonstrated to detect very subtle NADH differences with very high resolution, such as 12 micron resolution. It is expected that this technique can generate a metabolic map of the tumor that can be visualized to differentiate a tumor from the normal surrounding brain tissue. According to the best information available to the inventors, the delta doped CCD camera has never been used for brain tumor delineation before. It is believed that these systems and methods can also be used in the operating room to investigate the autofluorescent signature on a variety of different malignant human brain tumors. The types of cancer that the systems and methods of the invention are expected to be able to identify include Glioblastoma Multiforme, Asterocytomas and brain metastasis of cancers that originate elsewhere in the body.

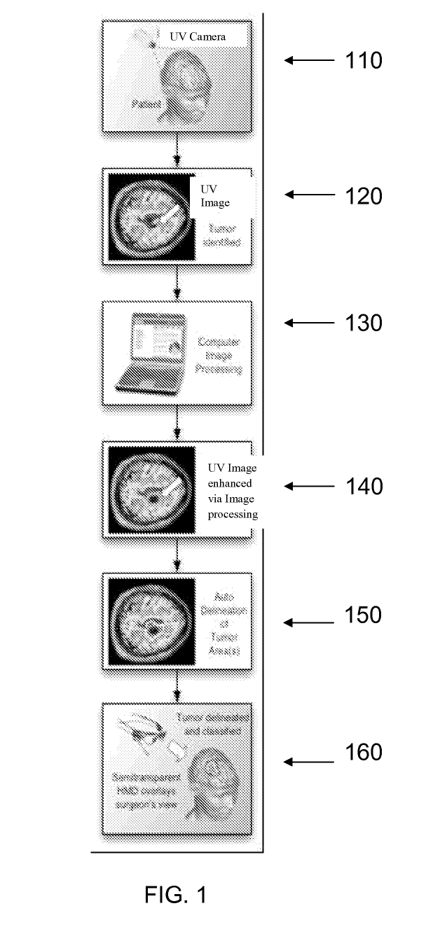

FIG. 1 is a schematic flow diagram of an imaging process according to principles of the invention. In step 110, the patient is prepared for brain surgery in the usual manner and a camera that employs the detector of the invention is positioned so as to observe the exposed portions of the patient's brain. The camera is set up so as not to interfere with the activity of medical personnel. In step 120, an UV image is obtained, which image can be used to identify the location and extent of a tumor. In sequence with the taking of the UV image, an image using visible illumination can also be recorded with the same camera if the filter is taken out, which visible illumination may be provided separately for the purposes of performing the surgical procedure. In step 130, the UV image obtained in step 120 is processed using a general purpose programmable computer that operates under the control of instructions recorded on a machine readable memory. In step 140, the UV image is enhanced using image processing techniques. In step 150, image processing techniques are used to delineate the area or areas that contain tumors. In step 150, a comparison of the UV image and the visible image can be performed. In some embodiments, the UV image alone may be sufficient to delineate the tumor if the signal from the tumor is sufficiently different from the signal from the normal tissue. The visual image can be used to display the extent of the tumor, for example, using an outline or as a false color region in the visual image. In step 160, after the tumor is delineated and classified, an image of the tumor can be presented to the medical personnel. In some embodiments, the image is presented to the medical personnel on a display. In other embodiments, the image is presented using special eyewear that allows sensing of the relative positions and orientations of the wearer and the camera relative to the field of operation so that an image of the proper region can be computed and presented to a viewer in at least one lens of the eyewear.

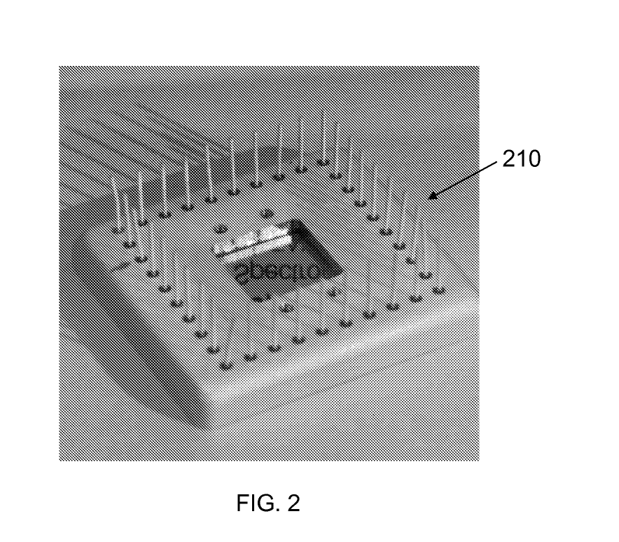

FIG. 2 is an image of a δ-doped CCD detector with a structurally supported membrane, which is advantageous for robustness and applications using fast optics. The δ-doped CCD detector is sensitive in the UV and in the visible. Various δ-doped CCDs have been described in U.S. Pat. Nos. 5,376,810, 6,403,963, 7,786,421 and 7,800,040 and in US Application Publication Nos. 2009/0116688, 2011/0140246, 2011/0169119, 2011/0304022, 2011/0316110, and 2012/0168891.

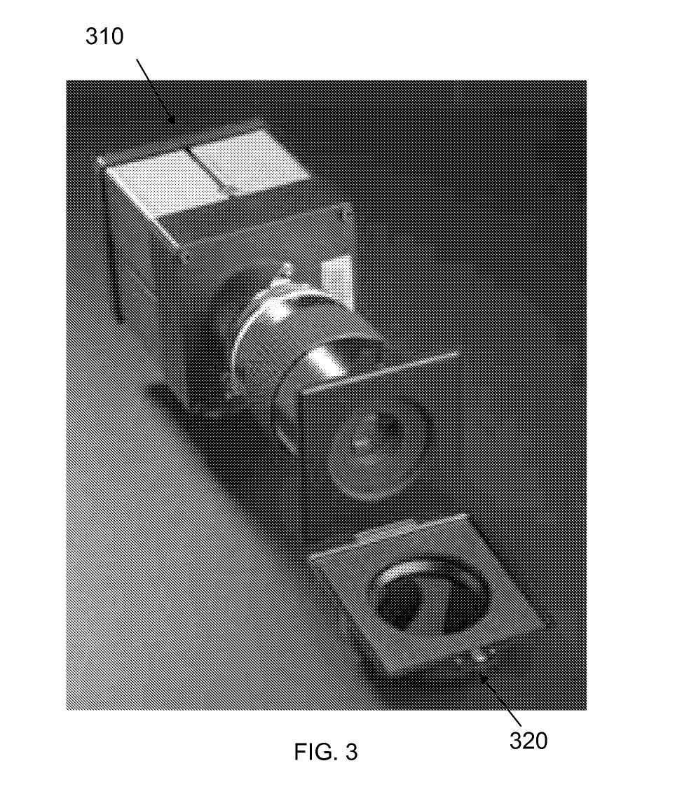

FIG. 3 is an image of a portable, high-frame rate UV/visible camera 210 that employs the δ-doped CCD detector of FIG. 2. The detector features 100% internal quantum efficiency. The detector does not exhibit hysteresis. The CCD in the camera has been shown to be stable for years. In one embodiment, the CCD provides 1024×1024 pixels having a 12 micron pixel size, with either frame transfer or full frame operation, at frame rates of 1-30 frames per second (which is the electronics capability), and 1-10 frames per second (which is the current chip capability). The camera can be operated under digital control and provides digital output. It is expected that future δ-doped CCDs and CMOS imaging detectors will have improved properties as compared to the present δ-doped CCD, such as more pixels, or such as a faster frame rate. The camera has a filter with a pass band in the UV, the filter being controllable to allow the camera to receive UV without visible illumination when the filter is engaged, and allowing the camera to receive visible illumination when the filter is removed from the optical path. The camera has at least one input port and at least one output port for communication with a computer. In one embodiment, the data from the UV/visible camera 210 is stored and processed in a general purpose programmable computer such as a laptop computer, which computer has access to instructions recorded on a machine readable medium, such that when the instructions are operating, the computer is programmed to operate the camera, record the data taken by the camera, process the data taken by the camera, and display results of such computations.

The camera can be provided with a demountable UV lens and visible-blind filter 220. The camera can operate at room temperature and can be used with thermoelectric cooling. The focus distance with current lenses can be as long as several meters. In some embodiments, the spectral range covers the UV and the visible, with a 300 nm short wavelength, due to lens cutoff and atmosphere absorption.

As is described hereinbelow in greater detail, the present invention contemplate the use of the UV/Visible Camera in the operating room to distinguish between tumorous and healthy tissue. In one embodiment, room light and/or UV LED illumination are used for excitation. Emission wavelength-selecting filters are placed in front of the camera to improve delineation. The detected emission signal is affected by the field of view, the detector efficiency at the given wavelength, the illumination intensity and light attenuation through air/optics. In particular, the use of a high quantum efficiency detector improves the signal strength as compared to less efficient detectors.

The camera has been tested at room temperature. A UV-Nikkor 105 mm f/4.5 lens that is made of fluorite and quartz glass was used. The Nikkor filter that was used has a transmission band centered on 330 nm, and transmits UV rays at wavelengths from 220 nm to 420 nm. The measured spectral transmittance is as high as 70%, ranging from 220 nm to 900 nm. The transmittance curve is flat. Manual focusing was performed by turning on room lights and focused accordingly, while checking the live image via Video Savant. Video Savant is high speed digital video recording software available from IO Industries Inc., 1615 North Routledge Park, Unit 27, London, ON, Canada N6H 5N5.

Several methods of providing excitation are possible. One embodiment uses regular fluorescent lights as are expected to be found in a typical operating room. Another embodiment involves using LEDs having a 385 nm (peak) and having a 405 nm (peak) for excitation. In some embodiments, the excitation will be in the range of 310 nm-415 nm. It is reported that the NADH absorption range is at 320 nm-380 nm, and that NADH emits fluorescent light at 420 nm-480 nm.

Filters tested for the excitation wavelengths included a Nikkor UV Filter (centered at 330 nm) was tested in the wavelength range of 310 nm-360 nm, and an Edmund Optics Filter (centered at 390 nm) was tested on in the wavelength range of 385 nm-415 nm. A Edmund Optics High Transmission OD 6 Bandpass Filter (centered at 472 nm) was tested in the emission wavelength range of interest. The filter has a 30 nm bandwidth and a transmission of greater than 93%, and it blocks light in the wavelength ranges of 250 nm-442 nm and 498 nm-640 nm.

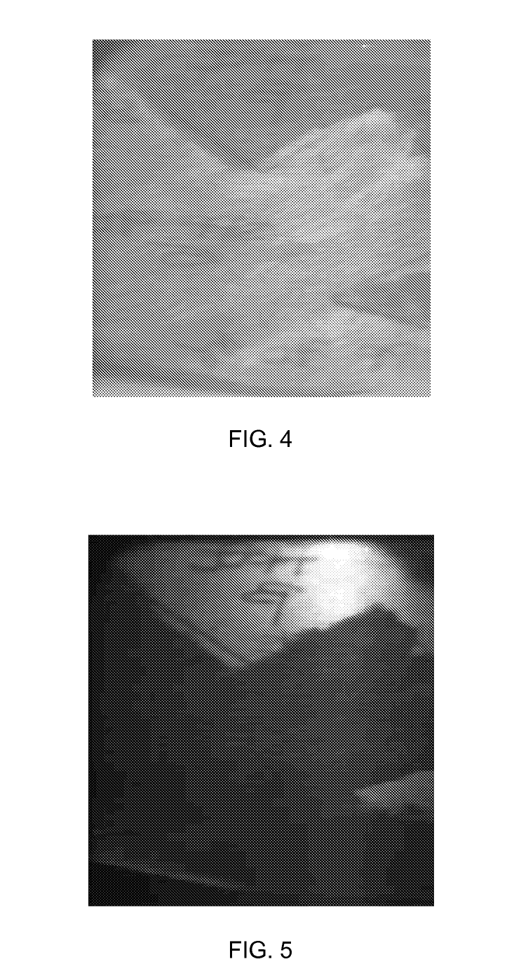

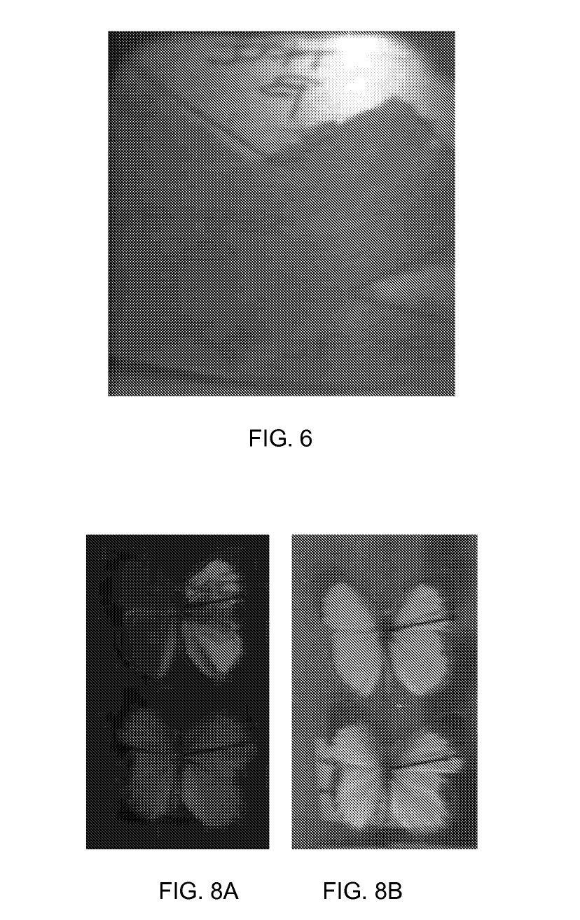

Various images have been recorded with the camera and filters described including those show in FIG. 4, FIG. 5 and FIG. 6.

FIG. 4 is an image of a piece of paper brushed with SPF 60 sunscreen recorded using room light as the illumination source.

FIG. 5 is an image of a piece of paper brushed with SPF 60 sunscreen recorded using a 385 nm LED as the illumination source.

FIG. 6 is an image of a piece of paper brushed with SPF 60 sunscreen recorded using both room light and a 385 nm LED as the combined illumination source.

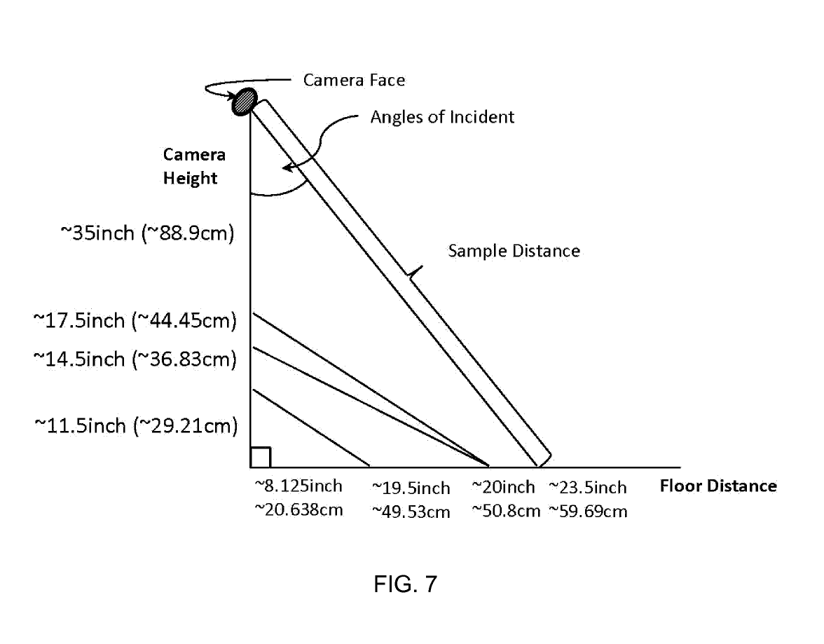

FIG. 7 is a diagram that illustrates the distance and angular relationships between the camera and the surface to be viewed and recorded. Table II describes a selected number of the relationships illustrated in FIG. 7 in numerical form.

The results shown in FIG. 8A and FIG. 8B demonstrate that the camera of the invention can be used to detect biological signatures that are not detected in the visible range. Images of Euremalisa male and female butterflies are used to demonstrate this point.

FIG. 8A illustrates Euremalisa butterflies imaged with the camera of FIG. 3 in ultraviolet.

FIG. 8B illustrates Euremalisa butterflies imaged with the camera of FIG. 3 in the visible.

As shown in the photographs, the UV image of the male butterfly (upper butterfly) shows higher reflectivity in the UV due to presence of proteins on the wings of the male butterfly. This effect is absent in the visible image. The female butterfly is the lower butterfly in each image.

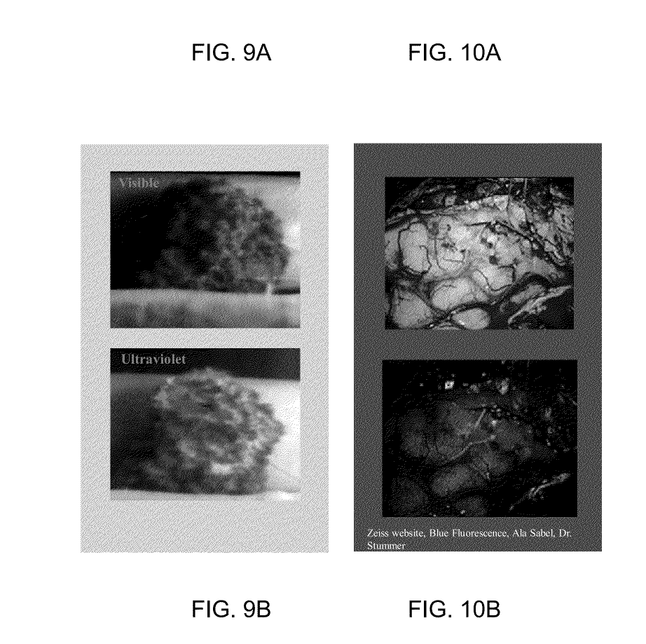

FIG. 9A illustrates a rock observed under visible light illumination.

FIG. 9B illustrates the rock of FIG. 9A observed under UV illumination. Regions of the rock that fluoresce can be seen.

FIG. 10A illustrates an exposed region of a brain observed under visible light illumination.

FIG. 10B illustrates the exposed region of a brain of FIG. 10A observed under UV illumination.

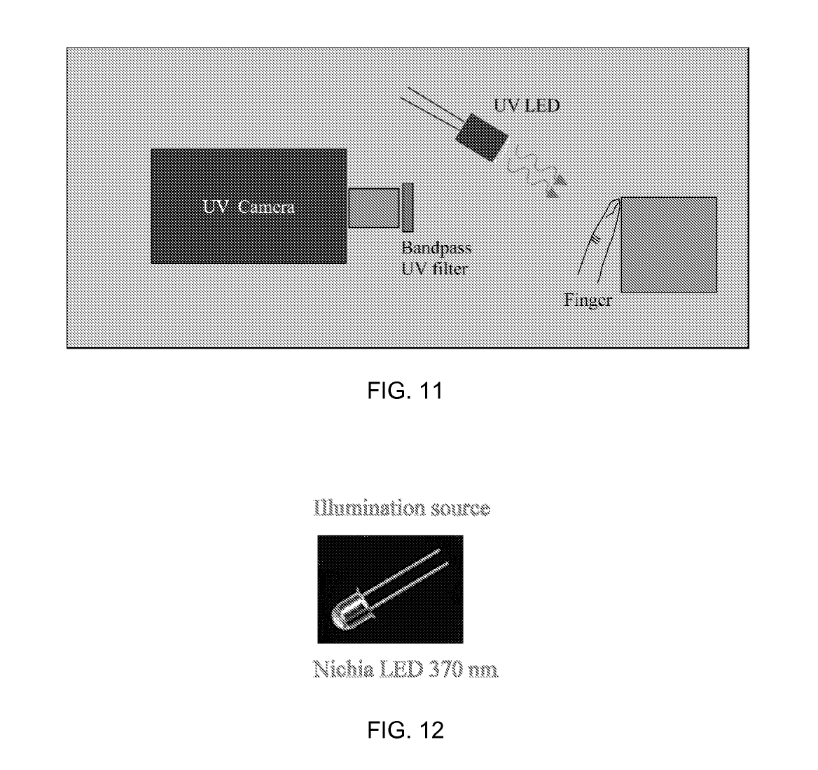

FIG. 11 is a schematic diagram of an experimental observation of a human finger under UV illumination.

FIG. 12 is an image of a UV LED.

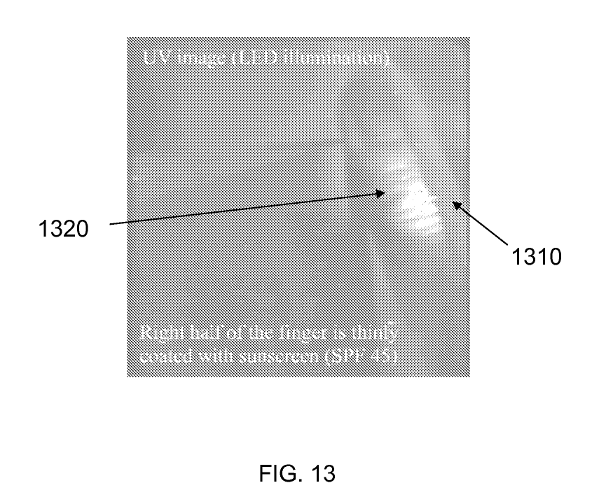

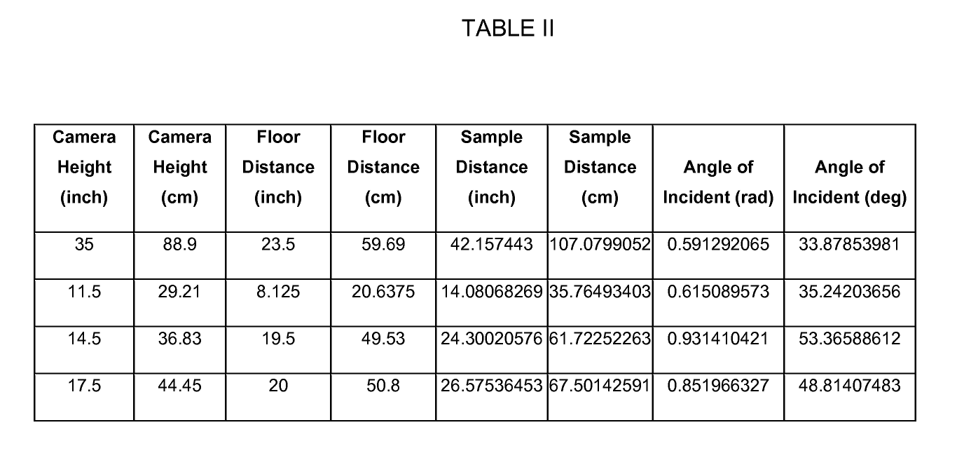

FIG. 13 is an image of a human finger observed under UV illumination conditions. In the image of FIG. 13, one portion, region 1310, of the finger has been coated with a thin layer of sunscreen (SPF 45) that absorbs UV, and the other portion, region 1320, of the finger has not been so treated. The response of the skin to the UV is apparent in region 1320.

### DEFINITIONS

Unless otherwise explicitly recited herein, any reference to an electronic signal or an electromagnetic signal (or their equivalents) is to be understood as referring to a non-volatile electronic signal or a non-volatile electromagnetic signal.

Recording the results from an operation or data acquisition, such as for example, recording results at a particular frequency or wavelength, is understood to mean and is defined herein as writing output data in a non-transitory manner to a storage element, to a machine-readable storage medium, or to a storage device. Non-transitory machine-readable storage media that can be used in the invention include electronic, magnetic and/or optical storage media, such as magnetic floppy disks and hard disks; a DVD drive, a CD drive that in some embodiments can employ DVD disks, any of CD-ROM disks (i.e., read-only optical storage disks), CD-R disks (i.e., write-once, read-many optical storage disks), and CD-RW disks (i.e., rewriteable optical storage disks); and electronic storage media, such as RAM, ROM, EPROM, Compact Flash cards, PCMCIA cards, or alternatively SD or SDIO memory; and the electronic components (e.g., floppy disk drive, DVD drive, CD/CD-R/CD-RW drive, or Compact Flash/PCMCIA/SD adapter) that accommodate and read from and/or write to the storage media. Unless otherwise explicitly recited, any reference herein to “record” or “recording” is understood to refer to a non-transitory record or a non-transitory recording.

As is known to those of skill in the machine-readable storage media arts, new media and formats for data storage are continually being devised, and any convenient, commercially available storage medium and corresponding read/write device that may become available in the future is likely to be appropriate for use, especially if it provides any of a greater storage capacity, a higher access speed, a smaller size, and a lower cost per bit of stored information. Well known older machine-readable media are also available for use under certain conditions, such as punched paper tape or cards, magnetic recording on tape or wire, optical or magnetic reading of printed characters (e.g., OCR and magnetically encoded symbols) and machine-readable symbols such as one and two dimensional bar codes. Recording image data for later use (e.g., writing an image to memory or to digital memory) can be performed to enable the use of the recorded information as output, as data for display to a user, or as data to be made available for later use. Such digital memory elements or chips can be standalone memory devices, or can be incorporated within a device of interest. “Writing output data” or “writing an image to memory” is defined herein as including writing transformed data to registers within a microcomputer.

“Microcomputer” is defined herein as synonymous with microprocessor, microcontroller, and digital signal processor (“DSP”). It is understood that memory used by the microcomputer, including for example instructions for data processing coded as “firmware” can reside in memory physically inside of a microcomputer chip or in memory external to the microcomputer or in a combination of internal and external memory. Similarly, analog signals can be digitized by a standalone analog to digital converter (“ADC”) or one or more ADCs or multiplexed ADC channels can reside within a microcomputer package. It is also understood that field programmable array (“FPGA”) chips or application specific integrated circuits (“ASIC”) chips can perform microcomputer functions, either in hardware logic, software emulation of a microcomputer, or by a combination of the two. Apparatus having any of the inventive features described herein can operate entirely on one microcomputer or can include more than one microcomputer.

General purpose programmable computers useful for controlling instrumentation, recording signals and analyzing signals or data according to the present description can be any of a personal computer (PC), a microprocessor based computer, a portable computer, or other type of processing device. The general purpose programmable computer typically comprises a central processing unit, a storage or memory unit that can record and read information and programs using machine-readable storage media, a communication terminal such as a wired communication device or a wireless communication device, an output device such as a display terminal, and an input device such as a keyboard. The display terminal can be a touch screen display, in which case it can function as both a display device and an input device. Different and/or additional input devices can be present such as a pointing device, such as a mouse or a joystick, and different or additional output devices can be present such as an enunciator, for example a speaker, a second display, or a printer. The computer can run any one of a variety of operating systems, such as for example, any one of several versions of Windows, or of MacOS, or of UNIX, or of Linux. Computational results obtained in the operation of the general purpose computer can be stored for later use, and/or can be displayed to a user. At the very least, each microprocessor-based general purpose computer has registers that store the results of each computational step within the microprocessor, which results are then commonly stored in cache memory for later use, so that the result can be displayed, recorded to a non-volatile memory, or used in further data processing or analysis.

Many functions of electrical and electronic apparatus can be implemented in hardware (for example, hard-wired logic), in software (for example, logic encoded in a program operating on a general purpose processor), and in firmware (for example, logic encoded in a non-volatile memory that is invoked for operation on a processor as required). The present invention contemplates the substitution of one implementation of hardware, firmware and software for another implementation of the equivalent functionality using a different one of hardware, firmware and software. To the extent that an implementation can be represented mathematically by a transfer function, that is, a specified response is generated at an output terminal for a specific excitation applied to an input terminal of a “black box” exhibiting the transfer function, any implementation of the transfer function, including any combination of hardware, firmware and software implementations of portions or segments of the transfer function, is contemplated herein, so long as at least some of the implementation is performed in hardware.

### Theoretical Discussion

Although the theoretical description given herein is thought to be correct, the operation of the devices described and claimed herein does not depend upon the accuracy or validity of the theoretical description. That is, later theoretical developments that may explain the observed results on a basis different from the theory presented herein will not detract from the inventions described herein.

Any patent, patent application, patent application publication, journal article, book, published paper, or other publicly available material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

While the present invention has been particularly shown and described with reference to the preferred mode as illustrated in the drawing, it will be understood by one skilled in the art that various changes in detail may be affected therein without departing from the spirit and scope of the invention as defined by the claims.

What is claimed is:

1. A medical imaging system, comprising:
a camera having a back illuminated silicon imaging detector that is sensitive in the visible and in the UV portions of the electromagnetic spectrum, said camera having a filter with a pass band in the UV, said filter being controllable to allow said camera to receive UV without visible illumination when said filter is engaged, and being controllable to allow said camera to receive visible illumination when said filter is removed from an optical path, said camera having at least one control port and having at least one output port;
as required if not already present in a location of use, a UV illumination source configured to illuminate the field of operation in a surgical procedure on a brain of a patient, said UV illumination source, if provided, controllable by a controller;
a controller configured to control an operation of said camera having said detector and said filter with said pass band in the UV; and
a general purpose programmable computer configured to receive output data from said at least one output port of said camera having said detector, said general purpose programmable computer having access to instructions recorded on a machine readable medium, such that when the instructions are operating, the computer is programmed to operate the camera, record the data taken by the camera, process the data taken by the camera, and display a result of such computation to a user of said medical imaging system.

2. The medical imaging system of claim 1, wherein said back illuminated silicon imaging detector is a detector selected from the group consisting of a δ-doped detector and a multilayer doped detector.

3. The medical imaging system of claim 1, wherein said back illuminated silicon imaging detector is a detector having a device structure selected from the group consisting of a CCD detector, a CMOS detector, a photodiode detector array, a hybrid photodiode detector array, and an avalanche photodiode detector array.

4. The medical imaging system of claim 1, wherein said back illuminated silicon imaging detector comprises an anti-reflection coating.

5. The medical imaging system of claim 1, wherein said back illuminated silicon imaging detector has at least 1024×1024 pixels.

6. The medical imaging system of claim 1, wherein said back illuminated silicon imaging detector is sensitive to illumination in the range of 420 nm-480 nm.

7. The medical imaging system of claim 1, wherein said UV filter blocks light in the wavelength ranges of 250 nm-442 nm and 498 nm-640 nm.

8. The medical imaging system of claim 1, wherein said controller is implemented in said general purpose programmable computer.
